(19) United States
(12) Patent Application Publication
(10) Pub. No.: US 2023/0108486 A1
(43) Pub. Date: Apr. 6, 2023

(54) CONCENTRATION-LEVEL ESTIMATION DEVICE, CONCENTRATION-LEVEL ESTIMATION METHOD, AND RECORDING MEDIUM

(71) Applicant: Panasonic Intellectual Property Management Co., Ltd., Osaka (JP)

(72) Inventors: Katsuhiro KANAMORI, Nara (JP); Souksakhone BOUNYONG, Osaka (JP); Kunihiro IMAMURA, Osaka (JP); Mototaka YOSHIOKA, Osaka (JP)

(21) Appl. No.: 17/907,900

(22) PCT Filed: Mar. 23, 2021
(86) PCT No.: PCT/JP2021/012091

§ 371 Date: Aug. 29, 2022

(30) Foreign Application Priority Data
Mar. 26, 2020 (JP) 2020-055449

(51) Int. Cl. G06V 40/20; G10L 25/51

(52) U.S. Cl. CPC G06V 40/20; G10L 25/51

(57) ABSTRACT

A concentration-level estimation device includes an obtainer which obtains task information indicating a type of a task executed by a user among types of tasks; a sensor which outputs motion information indicating a feature of a motion of the user who executes the task, based on a result of sensing obtained from a sensing device; a storage which stores profiles concerning habits of the user for the types of tasks; and a calculator which calculates a concentration level of the user using the motion information and a profile corresponding to the type of the task indicated in the task information among the profiles stored in the storage.

### CROSS-REFERENCE OF RELATED APPLICATIONS

This application is the U.S. National Phase under 35 U.S.C. § 371 of International Patent Application No. PCT/JP2021/012091, filed on Mar. 23, 2021, which in turn claims the benefit of Japanese Patent Application No. 2020-055449, filed on Mar. 26, 2020, the entire disclosures of which Applications are incorporated by reference herein.

### TECHNICAL FIELD

The present disclosure relates to a concentration-level estimation device, a concentration-level estimation method, and a recording medium for executing the concentration-level estimation method using a computer.

### BACKGROUND ART

Concentration-level estimation devices which calculate concentration levels of people are known in the related art. For example, the concentration-level estimation device disclosed in Patent Literature (PTL) 1 can precisely capture the concentration level of a user based on room environment information regarding an image of the user and a room environment in which the user is present.

### CITATION LIST

### Patent Literature

[PTL 1] Japanese Unexamined Patent Application Publication No. 2019-82311

### SUMMARY OF INVENTION

### Technical Problem

However, the concentration-level estimation device in the related art cannot appropriately calculate the concentration level of a user in some cases.

Thus, an object of the present disclosure is to provide a concentration-level estimation device which can appropriately calculate the concentration level, and the like.

### Solution to Problem

To solve the above problem, the concentration-level estimation device according to one aspect of the present disclosure includes an obtainer which obtains task information indicating a type of a task executed by a user among types of tasks; a sensor which outputs motion information indicating a feature of a motion of the user who executes the task, based on a result of sensing obtained from a sensing device; a storage which stores profiles concerning habits of the user for the types of tasks; and a calculator which calculates a concentration level of the user using the motion information and a profile corresponding to the type of the task indicated in the task information among the profiles stored in the storage.

Moreover, the concentration-level estimation method according to one aspect of the present disclosure includes obtaining task information indicating a type of a task executed by a user among types of tasks; outputting motion information indicating a feature of a motion of the user who executes the task, based on a result of sensing obtained from a sensing device; and calculating a concentration level of the user using the motion information and a profile corresponding to the type of the task indicated in the task information among profiles concerning habits of the user for the types of tasks.

Moreover, one aspect of the present disclosure can be implemented as a program for causing a computer to execute the concentration-level estimation method. Alternatively, it can also be implemented as a computer-readable recording medium on which the program is stored.

### Advantageous Effects of Invention

The present disclosure can appropriately calculate the concentration level.

### BRIEF DESCRIPTION OF DRAWINGS

FIG. 1 is a diagram illustrating a usage example of the concentration-level estimation device according to an embodiment.

FIG. 2 is a functional block diagram illustrating the concentration-level estimation device according to the embodiment and its peripheral devices.

FIG. 3 is a diagram illustrating the profiles stored in the storage according to the embodiment.

FIG. 4A is a first diagram illustrating a type of task according to the embodiment.

FIG. 4B is a diagram illustrating states of a user who is executing the task illustrated in FIG. 4A.

FIG. 5A is a second diagram illustrating a type of task according to the embodiment.

FIG. 5B is a diagram illustrating states of a user who is executing the task illustrated in FIG. 5A.

FIG. 6 is a first diagram illustrating a combined type of task according to the embodiment.

FIG. 7 is a second diagram illustrating a combined type of task according to the embodiment.

FIG. 8 is a flowchart showing the operation of the concentration-level estimation device according to the embodiment.

FIG. 9 is a block diagram illustrating a learning device for generating profiles according to the embodiment and its peripheral devices.

FIG. 10 is a diagram illustrating the concentration timing for generating profiles according to the embodiment.

FIG. 11 is a block diagram illustrating a functional configuration of the concentration-level estimation device according to Modification 1 of the embodiment.

FIG. 12 is a diagram illustrating profiles stored in the storage according to Modification 1 of the embodiment.

FIG. 13 is a block diagram illustrating a functional configuration of the concentration-level estimation device according to Modification 2 of the embodiment.

FIG. 14 is a diagram illustrating profiles stored in the storage according to Modification 2 of the embodiment.

### DESCRIPTION OF EMBODIMENT

### (Knowledge Underlying the Present Disclosure)

Traditionally, using captured images of a user, the concentration level, which is a degree of concentration of the user, is estimated (or calculated) and binarized. Recently, devices which more precisely calculate the concentration level of the user have been developed by considering other factors in addition of the images of the user. For example, PTL 1 discloses a concentration-level estimation device which improves precision of the concentration level of a user calculated using an image and room environment information concerning the room environment in which the user is present.

On the other hand, the user may make a significantly large variety of actions when concentrating, and a sensed action may indicate a concentrating state of one user, but may indicate a distracting state of another user in some cases. In particular, when the user is executing a task, the action which the user may make when concentrating can be varied according to the type of task. In other words, a user, who executes a task, may make one action when concentrating, while the user, who executes another task, may make another action when concentrating.

Thus, in the present disclosure, a concentration-level estimation device and the like which calculate the concentration level of the user in different methods for the types of tasks will be described. According to the present disclosure, the concentration level of the user can be appropriately estimated even when the action which the user may make when concentrating is varied for different types of tasks.

Hereinafter, the embodiment according to the present disclosure will be described with reference to the drawings. The embodiment described below illustrate comprehensive or specific examples of the present disclosure. Numeric values, shapes, materials, components, arrangement positions of components, connection forms thereof, steps, order of the steps, and the like shown in the embodiment below are exemplary, and should not be construed as limitations to the present disclosure. Among the components of the embodiment below, the components not described in an independent claim of the present disclosure are described as optional components.

The drawings are schematic views, and are not always exact illustrations. Accordingly, the scale is not consistent among the drawings. In the drawings, identical reference signs will be given to substantially identical configurations, and overlapping of description will be omitted or simplified.

### Embodiment

### [Configuration of Concentration-Level Estimation Device]

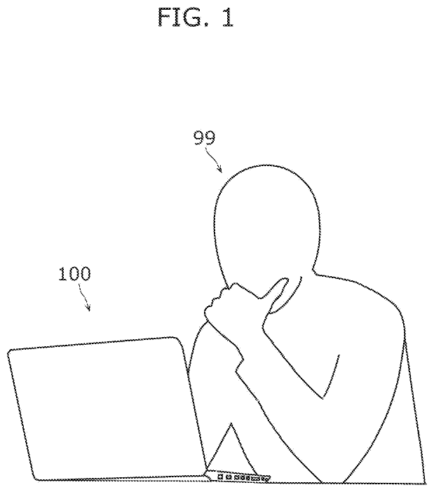

Initially, with reference to FIG. 1, the concentration-level estimation device according to an embodiment will be described. FIG. 1 is a diagram illustrating a usage example of the concentration-level estimation device according to the embodiment.

As illustrated in FIG. 1, concentration-level estimation device 100 according to the present embodiment is built in a computer or the like used by user 99 for implementation. By implementing concentration-level estimation device 100 built in a computer or the like used by user 99, peripheral devices mounted on the computer, such as an imaging device and a display, can be used. In particular, concentration-level estimation device 100 according to the present embodiment is used in a situation where user 99 is executing a task. Thus, when the task is implemented by a computer and an application executed on the computer, concentration-level estimation device 100 according to the present embodiment is suitable because the execution of the task and the calculation of the concentration level can be performed on a single computer.

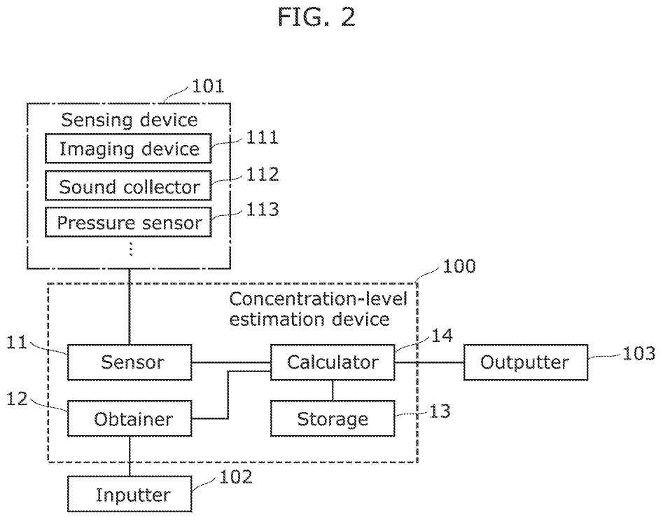

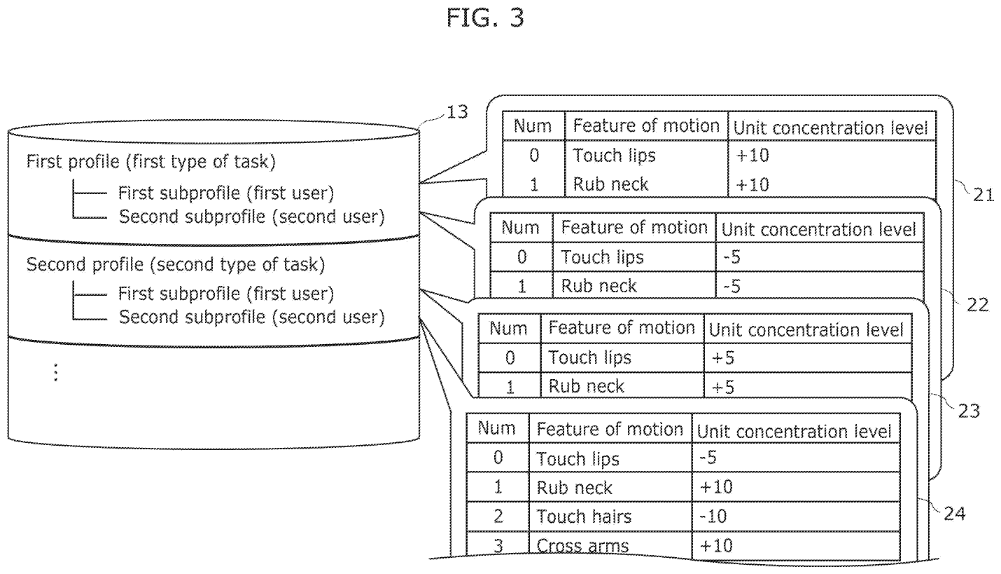

Next, with reference to FIGS. 2 and 3, the functional configurations of concentration-level estimation device 100 will be described. FIG. 2 is functional block diagram illustrating the concentration-level estimation device according to the embodiment and its peripheral devices. In FIG. 2, in addition to concentration-level estimation device 100, sensing device 101, inputter 102, and outputter 103 are shown as peripheral devices. Concentration-level estimation device 100 includes sensor 11, obtainer 12, storage 13, and calculator 14. Hereinafter, the components of concentration-level estimation device 100 will be described in association with the peripheral devices.

Sensing device 101 is a device which performs a variety of sensing operations on user 99 during calculation of the concentration level by concentration-level estimation device 100, and outputs the result of sensing. Specifically, sensing device 101 includes a variety of sensors which sense the action of user 99. For example, sensing device 101 includes imaging device 111, sound collector 112, and pressure sensor 113. Besides, sensing device 101 may include sensing devices such as an electromyograph, a sphygmograph, a sphygmomanometer, an eye tracker, a gyroscope sensing device, and a distance measurer. As above, sensing device 101 is configured with any combination of various sensors.

The result of sensing sensed by sensing device 101 is obtained by sensor 11 in concentration-level estimation device 100. Sensor 11 is a processor which generates the feature of a motion of user 99 based on the result of sensing, and is implemented by executing a program concerning the operation of sensor 11 using a processor and a memory.

For example, when imaging device 111 included in sensing device 101 captures an image, sensor 11 obtains the image output as a result of sensing, extracts the feature of the motion of user 99 from the image, and outputs the extracted feature. Specifically, based on the positional relation between two or more body portions of user 99 captured in the obtained image, by image recognition, sensor 11 specifies the posture of user 99 when the image is captured. Sensor 11 generates motion information indicating the feature of the motion of user 99 from the specified posture, and outputs the motion information. The output motion information is transmitted to calculator 14.

For example, when sound collector 112 included in sensing device 101 collects sounds, sensor 11 obtains a voice signal output as a result of sensing, extracts the feature of the motion of user 99 from the voice signal, and outputs the extracted feature. Specifically, using a high-pass filter, a low-pass filter, and a bandpass filter, sensor 11 specifies signal components of sounds at predetermined frequencies cyclically repeated. Sensor 11 specifies the signal components caused by the motion of user 99 from the specified signal components, generates motion information indicating the feature of the motion, and outputs the generated motion information. The output motion information is transmitted to calculator 14.

For example, when pressure sensor 113 included in sensing device 101 senses pressure, sensor 11 obtains a pressure distribution output as a result of sensing, extracts the feature of the motion of user 99 in the pressure distribution, and outputs the feature. Specifically, sensor 11 specifies the body movement of user 99 from transition of the pressure distribution or the like. Sensor 11 generates motion information indicating the feature of the motion of user 99 from the specified body movement of user 99, and outputs the feature. The output motion information is transmitted to calculator 14.

For each of other sensing devices (not illustrated) included in sensing device 101, similarly, sensor 11 generates motion information indicating the feature of the motion of user 99 from the result of sensing, outputs the motion information, and transmits the motion information to calculator 14.

Inputter 102 is a device for inputting information indicating the type of the task executed by user 99. For example, the type of the task is input by user 99 through inputter 102 before user 99 starts execution of the task. In this case, inputter 102 is implemented as an input device such as a keyboard, a touch pad, a mouse, or switches disposed for types of tasks. For example, when the task to be executed is a program executed on a computer, by cooperation of the program with concentration-level estimation device 100, the type of the task can be input without intervention of user 99. In this case, inputter 102 is implemented by preliminarily incorporating the function as inputter 102 in the program such that the information indicating the type of the task is output to concentration-level estimation device 100 at the start of execution of the program.

The type of task can be set with any number of 2 or more according to the number of profiles stored in storage 13 described later. For example, when learning tasks for an educational purpose are assumed, the types of the tasks may be set for subjects such as Japanese language, science, mathematics, social sciences, and foreign language, or tasks for Japanese language may be set for scenes such as those in which user 99 listens to a lecture by a teacher, those in which user 99 works out tasks, those in which user 99 takes an examination, and those in which user 99 understands the contents of description by reading aloud or in silence. Similarly, types of tasks for which calculation of the concentration level is desired, such as business tasks, domestic tasks, and tasks for driving vehicles, are assumed, and their types can be set.

Hereinafter, among learning tasks, two types of tasks, which are tasks accompanied by an active action of user 99 and those accompanied by a passive action thereof, are assumed as the types of tasks, and the embodiment will be described. In other words, in the present embodiment, the types of tasks include a first type accompanied by an active action of user 99 when executing the task, and a second type accompanied by a passive action of user 99 when executing the task. The term “active” indicates the type of the task to which a response from user 99 is essential when the task is executed, and the term “passive” indicates the type of the task to which a response from user 99 is not essential when the task is executed.

Obtainer 12 is a processor which obtains information indicating the type of task from inputter 102, and is implemented by executing a program concerning the operation of obtainer 12 using a processor and a memory. More specifically, obtainer 12 obtains task information indicating which type of task user 99 executes among a plurality of types preliminarily set (here, two types). The task information is generated in inputter 102, and is transmitted to obtainer 12. Obtainer 12 converts the obtained task information to a format processable in calculator 14 described later, and transmits the converted task information to calculator 14.

Outputter 103 is a device which outputs the result of the concentration level calculated by calculator 14 and presents the result to user 99. For example, outputter 103 displays the calculated concentration level as an image on a display device included in a computer. Because the concentration level is calculated as a numeral which can be read out, outputter 103 may be implemented as a loudspeaker, and the calculated concentration level may be read out by the loudspeaker to present the concentration level to user 99, or may be read out by the loudspeaker and displayed as an image.

Storage 13 is a memory device which stores a variety of programs for implementing concentration-level estimation device 100, such as a semiconductor memory. Storage 13 stores profiles used in calculation of the concentration level as described above. Here, the profiles are related to habits of user 99. When the feature of the motion of user 99 indicated in the motion information matches with a motion which user 99 may make due to his/her habit, it can be determined that user 99 is more concentrating. The profiles are set for the types of tasks in a one-to-one correspondence. In other words, different habits of a user according to the types of tasks are stored in storage 13 as profiles for the respective types of tasks.

FIG. 3 is a diagram illustrating the profiles stored in the storage according to the embodiment. As illustrated in FIG. 3, storage 13 stores a first profile and a second profile corresponding to a first type of task and a second type of task, respectively. The first profile and the second profile each contain a first subprofile used in calculation of the concentration level of a first user contained in a first classification among users 99. Similarly, the first profile and the second profile each contain a second subprofile used in calculation of the concentration level of a second user contained in a second classification different from the first classification among users 99. The classification of users 99 including the first classification and the second classification is a concept meaning groups of users 99 classified based on similarities of habits. Examples of the classifications include a group of users who scratch their heads when they are concentrating, a group of uses who cross their arms when they are concentrating, and a group of users who tap desks with fingers when they are distracting.

By using such classifications of users 99, concentration-level estimation device 100 can appropriately calculate the concentration level based on not only the type of task but also the classifications of users 99. As above, each of the profiles may be further divided into lower profiles. Among a plurality of profiles including the first profile and the second profile, only one profile may be divided into a first subprofile and a second subprofile, and the remaining profiles may not be divided into subprofiles.

As illustrated in FIG. 3, in each of the profiles stored in storage 13, the feature of each motion which user 99 may make when concentrating due to a habit is associated with the unit concentration level indicating the concentration level of the feature of the motion. For example, in first subprofile 21 of the first profile, the unit concentration level “+10” is associated with “touch lips” as a feature of a motion when user 99 is concentrating. Similarly, in first subprofile 21 of the first profile, “+10” is associated with “rub neck”.

In some cases, even the feature of the same motion may be different between the first user and the second user, and may correspond to a motion when concentrating or a motion when distracting. For example, in first subprofile 21 of the first profile, “+10” is associated with “touch lips” while in second subprofile 22 of the first profile, “−5” is associated with “touch lips”.

In some cases, even for the feature of the same motion, the concentration level may be different between the first type of task and the second type of task. For example, in first subprofile 21 of the first profile, “+10” is associated with “touch lips” while in first subprofile 23 of the second profile, “+5” is associated with “touch lips”. For the feature of the same motion, the concentration level is equal in the first type of task and the second type of task. For example, “−5” is associated with “touch lips” in second subprofile 22 of the first profile and in second subprofile 24 of the second profile.

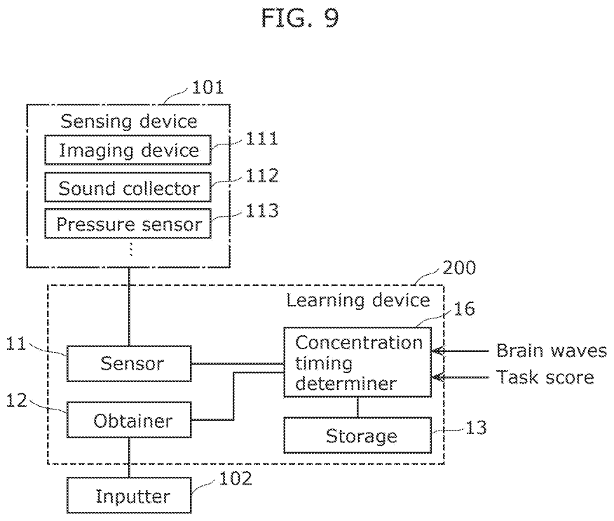

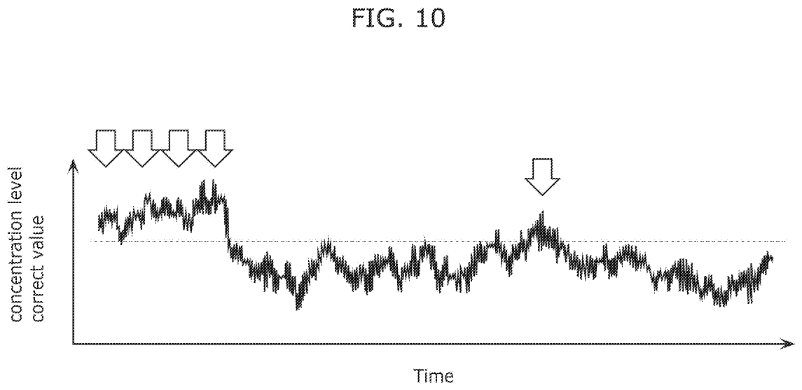

Learning device 200 (see FIG. 9 described later) which stores the profiles in storage 13 will be described later with reference to FIGS. 9 and 10.

Calculator 14 is a processor which calculates the concentration level of user 99 from the motion information received from sensor 11 and the task information received from obtainer 12 while referring to appropriate profiles stored in storage 13. Calculator 14 is implemented by executing a program concerning the operation of calculator 14 using a processor and a memory.

Depending on the type of task executed, which is indicated in the task information, calculator 14 reads out its associated profile (and subprofile) from storage 13. The read profile is associated with the feature of the motion which user 99 who is executing the type of task may make due to his/her habit and the unit concentration level.

For the feature of the motion of user 99 indicated in the motion information, calculator 14 calculates the concentration level of user 99 by adding the unit concentration levels associated in the profile corresponding to the type of task indicated in the task information. Specifically, for example, when it is determined from the task information that the task executed by the second user is of the second type, calculator 14 reads out second subprofile 24 of the second profile. Calculator 14 determines the motions being made by the second user from the received motion information, i.e., that the second user crosses the arms, and sometimes rubs the neck, and calculates the concentration level of the second user as “+20” from +10+10=+20.

As above, calculator 14 calculates the concentration level of user 99 using the profile corresponding to the type of task indicated in the task information and the motion information.

### [Active Task]

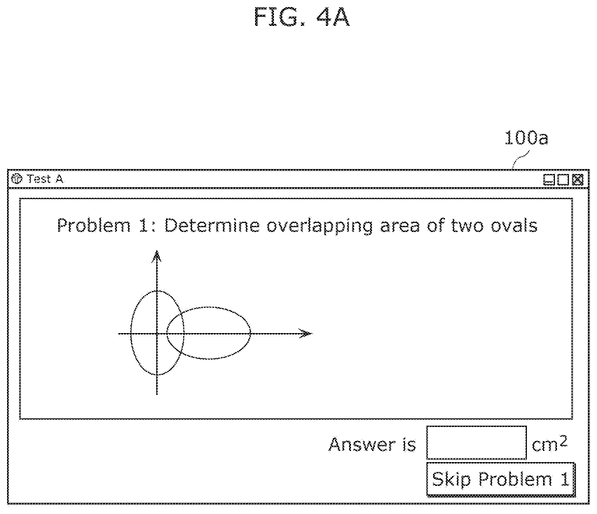

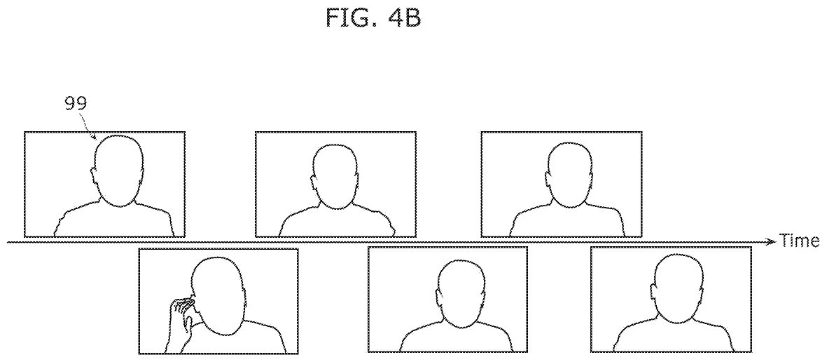

The first type of task accompanied by an active action of user 99 described above will be described. FIG. 4A is a first diagram illustrating a type of task according to the embodiment. FIG. 4B is a diagram illustrating states of the user who is executing the task illustrated in FIG. 4A.

FIG. 4A illustrates one example of the first type of task (in other words, active task), in which user 99 is required to execute calculation as an active action. As illustrated in FIG. 4A, on the GUI displayed on the display of a computer used by user 99, the first type of task prompts user 99 to display the content of the calculation problem and the answer input foam, and to input the result of the calculation problem solved by user 99 to the answer input foam.

In FIG. 4B, the states of user 99 captured by imaging device 111 disposed in an upper side of the display are aligned on a time-series basis. As illustrated in FIG. 4B, user 99 who is executing the first type of task basically solves the calculation problem while watching the display. Thus, the distance between the display and user 99 is kept substantially constant, and the posture does not significantly change. When user 99 is executing such a first type of task, user 99 does not significantly change the posture. For this reason, it is preferred that the habits appearing in detail can be sensed by sensing device 101. Examples of such habits include eye movements and convergence of user 99, furrows in the forehead, lip movements, and sounds and muscle movements when user is fiddling a writing tool or the like by fingers. Sensors which can sense these may be selected and arranged.

### [Passive Task]

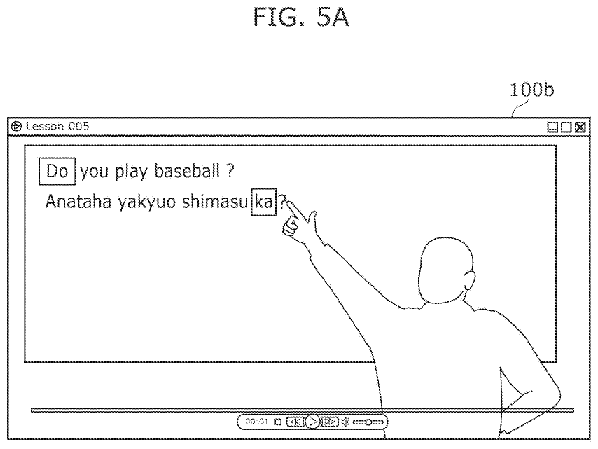

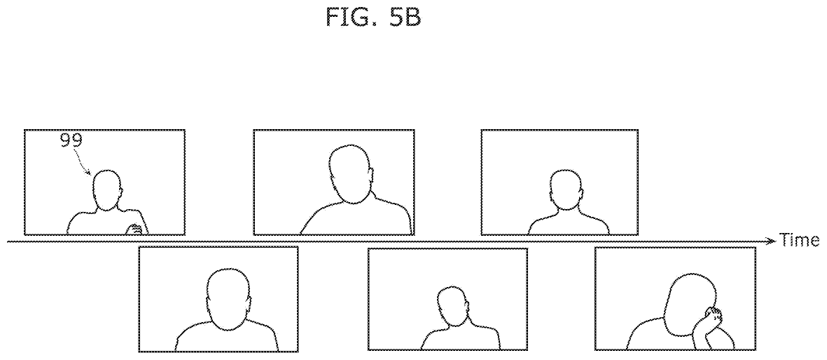

The second type of task accompanied by the passive action of user 99 described above will be described. FIG. 5A is a second diagram illustrating a type of task according to the embodiment. FIG. 5B is a diagram illustrating states of the user who is executing the task illustrated in FIG. 5A.

FIG. 5A illustrates one example of the second type of task (in other words, passive task), in which user 99 is required, as a passive action, to view a video taken in advance and learn without initiating any action from user 99, in other words, to execute viewing of a lecture given in the form of a moving picture. As illustrated in FIG. 5A, for the second type of task, user 99 simply views the lecture in the form of a moving picture reproduced on the GUI displayed on the display of the computer used by user 99.

In FIG. 5B, the states of user 99 captured by imaging device 111 disposed in an upper side of the display are aligned on a time-series basis. As illustrated in FIG. 5B, user 99 who is executing the second type of task views the display, or mainly listens to sounds while user 99 is away from the display, so that the distance between the display and user 99 is varied and the posture significantly changes. When user 99 is executing such a second type of task, user 99 significantly changes the posture. For this reason, it is preferred that such a change in posture and the habits of moving body parts largely can be sensed by sensing device 101. Examples of such habits include crossed arms of user 99, chin resting on hand, fixture of the posture, back and forth/left and right movements of the body, tilting of the head, and the appearance of drowsiness (such as yarn and the number of blinks), and sensors which can sense those habits may be selected and arranged.

### [Combination Type of Task]

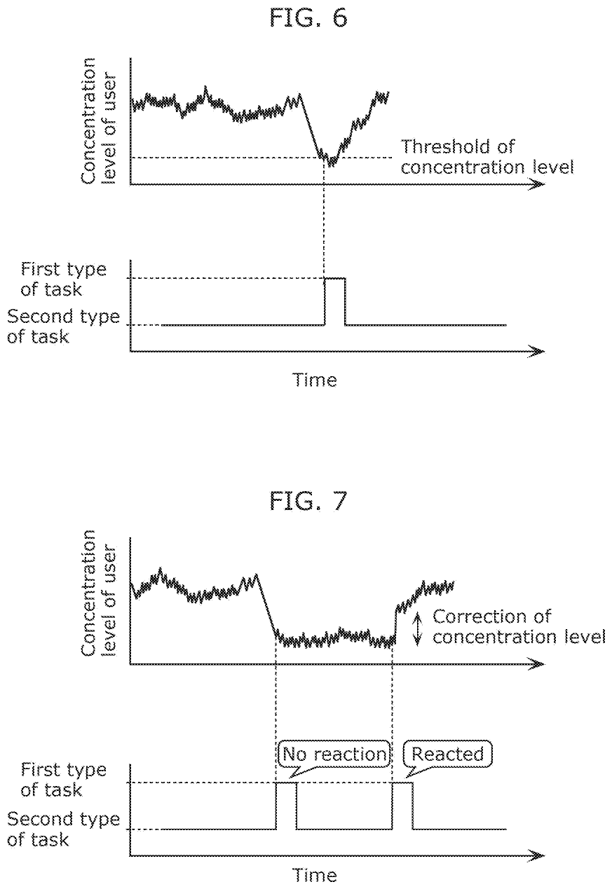

In addition to the first type of task and the second type of task described above, a combined type of task combined in a time-sharing manner can also be executed. In the combined type of task, for example, a reduction in concentration level of user 99 can be suppressed and the concentration level of user 99 can be corrected by executing the tasks concurrently with the calculation of the concentration level of user 99. Hereinafter, this configuration will be described with reference to FIGS. 6 and 7.

FIG. 6 is a first diagram illustrating the combined type of task according to the embodiment. The upper portion of FIG. 6 shows the transition of the concentration level of user 99 being calculated concurrently with the execution of the tasks, and the lower portion thereof shows a transition sequence diagram of the task type between the first type and the second type.

As illustrated in FIG. 6, in the combined type of task, when the concentration level of user 99 becomes lower than a predetermined threshold of the concentration level when the second type of task is being executed, the task transits to the first type of task. Thereby, the concentration level can be improved by transiting the task to the first type of task and prompting the active action of user 99 when the task efficiency is considered to be reduced due to the concentration level reduced during viewing of the lecture given in the moving picture or the like.

In the first type of task here, for example, a video taken in advance is reproduced in which a lecturer giving the lecture in the moving picture calls the name of user 99, and in response to the call, user 99 performs an operation, e.g., clicks on the GUI displayed as a pop-up window or the like on the screen.

In the first type of task here, for example, a video taken in advance may be reproduced in which the lecturer giving the lecture in the moving picture calls the name of user 99, and the video may contain a content that user 99 is requested to reply with a motion. In this case, the active action of user 99 is prompted simply by a reply to the call with a motion of user 99, such as nodding and responding.

Thus, in the combined type of task, it is expected that the concentration level can be improved again when user 99 is distracting.

FIG. 7 is a second diagram illustrating the combined type of task according to the embodiment. Similarly to FIG. 6, the upper portion of FIG. 7 shows the transition of the concentration level of user 99 calculated concurrently with the execution of the task, and the lower portion thereof shows a transition sequence diagram of the task type between the first type and the second type.

As illustrated in FIG. 7, in the combined type of task, a content of the first type of task is incorporated in the middle of the second type of task at a present timing. In other words, the task executed by user 99 transits to the first type of task at a predetermined timing when user 99 is executing the second type of task. Concentration-level estimation device 100 obtains the reaction of user 99 at the timing when the task transits to the first type of task, and corrects the concentration level calculated according to the presence/absence of the reaction.

The reaction is obtained by sensor 11 through sensing device 101. In other words, sensor 11 obtains a result of sensing for outputting the motion information, and in addition, further obtains a result of sensing for outputting the reaction information according to the reaction of user 99. The signal transmitted from sensing device 101 is the same in the motion information and the reaction information.

For this reason, sensor 11 processes the result of sensing concerning the reaction information, the result of sensing being obtained within a predetermined period set in consideration of a standard reaction time of people with reference to the transition timing to the first type of task, and outputs the reaction information. Concentration-level estimation device 100 may separately include sensor 11 which obtains a result of sensing concerning the motion information and a processor having a function which obtains a result of sensing concerning the response information.

The term “reaction” here means that user 99 replies with the voice and action of user 99, such as responding and nodding, or performs an operation, e.g., clicks on the GUI displayed as a pop-up window or the like on the screen. In the first type of task here, as described above, the lecturer calls the name of user 99, and a video taken in advance is reproduced, for example. In concentration-level estimation device 100, the reaction of user 99 who replies to this is obtained as a result of sensing from sensing device 101 including imaging device 111 and sound collector 112.

When a reaction to the call in the first type of task transited is absent, it is estimated that user 99 is distracting. For this reason, a low concentration level of user 99 calculated due to a habit which user 99 may make when distracting while executing the task is considered as correct.

In contrast, when a reaction to the call in the first type of task transited is present, it is estimated that user 99 is concentrating. For this reason, a low concentration level of user 99 calculated due to a habit which user 99 may make when distracting during executing the task is considered as incorrect. Calculator 14 corrects the concentration level by the unit concentration level associated with the incorrect feature of the motion of user 99 when distracting in the profile used for calculation of the concentration level, and calculates a higher concentration level. The degree of correction is determined according to the reaction speed of user 99, for example. This incorrect feature of the motion is the feature of the motion associated with the lowest unit concentration level or the feature of the motion associated with a relatively low unit concentration level in the profile used for calculation of the concentration level. The selection of the incorrect feature of the motion is one example, and may be selected with any other reference.

At this time, calculator 14 may correct the unit concentration level associated with the profile as the feature of the motion of user 99 when distracting, which is incorrect. Thereby, the profile is updated in the subsequent processing such that the concentration level is more appropriately calculated.

In the same manner as above, calculator 14 corrects a high concentration level of user 99 calculated due to a habit which user 99 may make when concentrating according to the presence/absence of the reaction in the first type of task, and calculates a lower concentration level.

Specifically, when a reaction to the call in the first type of task transited is absent, it is estimated that user 99 is distracting. For this reason, a high concentration level of user 99 calculated due to a habit which user 99 may make when concentrating while executing the task is considered as incorrect. Calculator 14 corrects the concentration level by the unit concentration level associated with the incorrect feature of the motion of user 99 when concentrating in the profile used for calculation of the concentration level, and calculates a lower concentration level. In this correction, processing is performed, for example, where the unit concentration level associated with the incorrect feature of the motion of user 99 when concentrating is 0. The incorrect feature of the motion is the feature of the motion associated with the highest unit concentration level or the feature of the motion associated with a relatively high unit concentration level in the profile used for calculation of the concentration level. The selection of the incorrect feature of the motion is one example, and may be selected with any other reference.

At this time, calculator 14 may correct the unit concentration level associated with the profile as the feature of the motion of user 99 when concentrating, which is incorrect. Thereby, the profile is updated in the subsequent processing such that the concentration level is more appropriately calculated.

### [Operation of Concentration-Level Estimation Device]

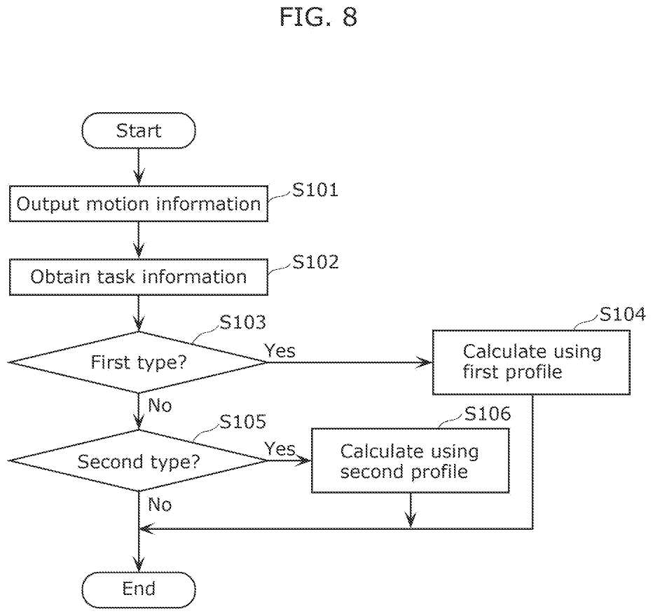

Next, the operation of concentration-level estimation device 100 described above will be described with reference to FIG. 8. FIG. 8 is a flowchart illustrating the operation of the concentration-level estimation device according to the embodiment.

As illustrated in FIG. 8, initially, based on the result of sensing obtained from sensing device 101, sensor 11 outputs the motion information (sensing step S101). The output motion information is received by calculator 14, and is used to calculate the concentration level.

In the next step, obtainer 12 obtains the task information indicating the type of the task executed by user 99 among types of tasks (obtaining step S102). Sensing step S101 and obtaining step S102 may be performed in the inverted order, or may be performed in parallel. The obtained task information is received by calculator 14, and is used to calculate the concentration level.

In the next step, calculator 14 determines whether the type of task indicated in the task information is the first type (first determination step S103). When the type of task is the first type (Yes in step S103), calculator 14 calculates the concentration level of user 99 using the first profile corresponding to the first type and the motion information (first calculating step S104).

In contrast, when the type of task is not the first type (No in first determination step S103), calculator 14 determines whether the type of task indicated in the task information is the second type (step S105). When the type of task is the second type (Yes in second determination step S105), calculator 14 calculates the concentration level of user 99 using the second profile corresponding to the second type and the motion information (second calculating step S106).

In contrast, when the type of task is not the second type (No in second determination step S105), concentration-level estimation device 100 terminates the processing. Although a case where as the types of tasks, two types, i.e., the first type and the second type have been described here, the types of tasks may be three or more types. For example, when there are N (where N is a natural number) types of tasks, calculator 14 sequentially performs a first determination step and a first calculating step, a second determination step and a second calculating step, a third determination step and a third calculating step, and so on to the N-th determination step and the N-th calculating step. Hereinafter, the first to N-th determination steps are collectively referred to as determination step, and the first to N-th calculating steps are collectively referred to as calculating step.

When a plurality of classifications of users 99 are presented as subprofiles, calculator 14 determines the classification of user 99 after every determination step, and calculates using the subprofile of the classification corresponding to the classification according to the result of determination. For example, when Yes in first determination step S103, calculator 14 determines whether the classification of user 99 is the first classification. When user 99 belongs to the first classification, calculator 14 calculates the concentration level of user 99 using first subprofile 21 in the first profile. Similarly, when user 99 belongs to the second classification rather than the first classification, calculator 14 calculates the concentration level of user 99 using second subprofile 22 in the first profile.

### [Learning Device]

Hereinafter, a device for generating the profiles by learning and storing the profiles in storage 13 will be described with reference to FIGS. 9 and 10. FIG. 9 is a block diagram illustrating a learning device for generating the profile according to the embodiment and its peripheral devices. FIG. 10 is a diagram illustrating the concentration timing for generating the profile according to the embodiment.

Learning device 200 illustrated in FIG. 9 has a substantially identical configuration with that of concentration-level estimation device 100. Thus, hereinafter, different configurations will be mainly described, and description of substantially identical configurations will be omitted or simplified.

As illustrated in FIG. 9, learning device 200 includes concentration timing determiner 16 instead of calculator 14. Concentration timing determiner 16 is connected to an electroencephalograph (not illustrated) attached to user 99, a counter (not illustrated) which gives a score to the task executed by user 99, and the like. Concentration timing determiner 16 is a processor which determines the timing when user 99 is concentrating, based on an index about the concentration level of user 99 obtained from the electroencephalograph, the counter, and the like. Concentration timing determiner 16 is implemented by executing a program concerning the operation of concentration timing determiner 16 using a processor and a memory.

As one example, from the electroencephalograph, concentration timing determiner 16 obtains brain waves of user 99 who is executing the task. As illustrated in FIG. 10, for example, the obtained brain waves oscillate along the temporal axis, and are used as a concentration level correct value indicating that a higher frequency thereof indicates a higher concentration level of user 99. The concentration level correct value when user 99 is sufficiently concentrating is preset as a threshold of concentration as shown with the dashed line. As shown with the arrows in the diagram, concentration timing determiner 16 determines a timing exceeding the threshold of concentration as a timing when user 99 is concentrating. The brain waves contain a large amount of noise components. To remove such noise components, concentration timing determiner 16 determines only the timings exceeding the threshold of concentration for a predetermined period as the timing when user 99 is concentrating.

Although not illustrated, concentration timing determiner 16 may determine the timing when user 99 is distracting, using a threshold of distraction preset in the concentration level correct value when user 99 is sufficiently distracting.

Concentration timing determiner 16 generates a profile corresponding to the type of the task executed by user 99, which is received from obtainer 12, and stores the profile in storage 13. Concentration timing determiner 16 updates the profile stored in storage 13 in association with the unit concentration level and the feature of the motion of user 99 indicated in the motion information received from sensor 11 at a timing when user 99 is concentrating. At this time, for example, the unit concentration level is set according to a degree how much the concentration level correct value exceeds the threshold of concentration. Thus, concentration-level estimation device 100 is configured using storage 13 which stores profiles. Learning device 200 can be implemented only by adding concentration timing determiner 16 to concentration-level estimation device 100, and concentration-level estimation device 100 also serving as learning device 200 can also be implemented.

### [Modification 1]

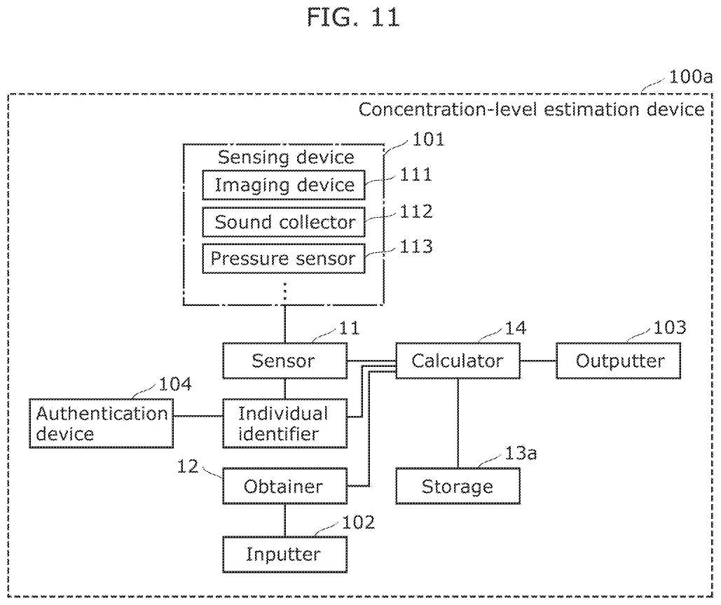

Hereinafter, modifications according to the embodiment will be described. FIG. 11 is a block diagram illustrating the functional configuration of the concentration-level estimation device according to Modification 1 of the embodiment.

In Modification 1, unlike concentration-level estimation device 100 described above, concentration-level estimation device 100a includes sensing device 101, inputter 102, and outputter 103 as the components. In other words, compared to concentration-level estimation device 100 described above, concentration-level estimation device 100a according to Modification 1 is an independent device which completes the operation without peripheral devices and the like. In other words, it can also be said that concentration-level estimation device 100 according to the embodiment above is a functional module which imparts a concentration-level estimation function as one function to a variety of apparatuses.

As illustrated in the drawing, unlike concentration-level estimation device 100, concentration-level estimation device 100a includes authentication device 104 and individual identifier 15 connected to the authentication device. Individual identifier 15 is a processor which identifies user 99 as a specific user, and is implemented by executing a program concerning the operation of individual identifier 15 using a processor and a memory. Individual identifier 15 obtains the authentication information by a specific user from authentication device 104, and identifies user 99 as the specific user using the authentication information.

More specifically, using a fingerprint authentication device or a login form using an ID and a password, authentication device 104 identifies which user among users 99 registered in a database (not illustrated) corresponds to the user who uses concentration-level estimation device 100a. Using the authentication information indicating the specific user identified by authentication device 104, individual identifier 15 identifies the user who uses concentration-level estimation device 100a, as the specific user.

Individual identifier 15 may include an authentication database of its own independent from authentication device 104. For example, individual identifier 15 may obtain an image of the user who uses concentration-level estimation device 100a, through sensor 11 from imaging device 111 included in sensing device 101, and may identify the specific user by checking the user against the authentication database of its own. In this case, concentration-level estimation device 100a does not need to include authentication device 104.

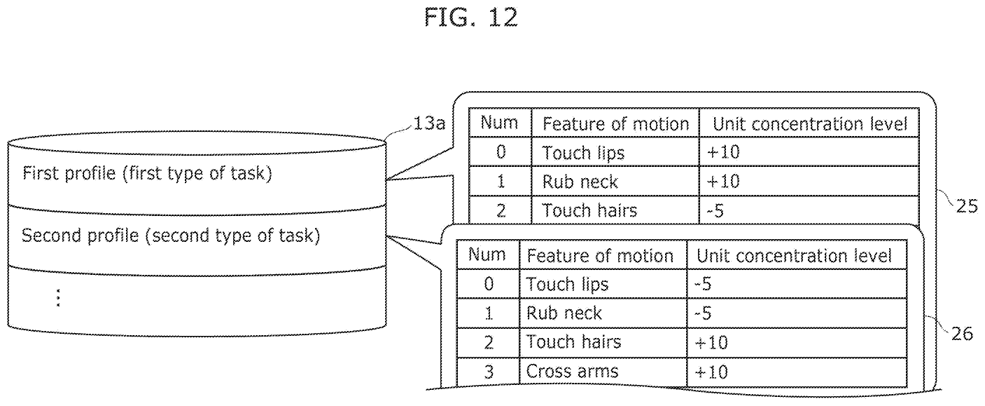

Thus, by identifying the specific user among users 99, concentration-level estimation device 100a unique to the specific user can be implemented using profiles specific to the specific user. FIG. 12 is a diagram illustrating the profiles stored in the storage according to Modification 1 of the embodiment. As described above, the profiles stored in storage 13a of concentration-level estimation device 100a contain profiles concerning habits of the specific user for each type of task.

In other words, as illustrated in FIG. 12, storage 13a contains first specific profile 25 used to calculate the concentration level of the specific user when the specific user executes the first type of task, and second specific profile 26 used to calculate the concentration level of the specific user when the specific user executes the second type of task. The operation of concentration-level estimation device 100a is the same as that of concentration-level estimation device 100 above except that the user is a specific user, and thus the description thereof will be omitted.

### [Modification 2]

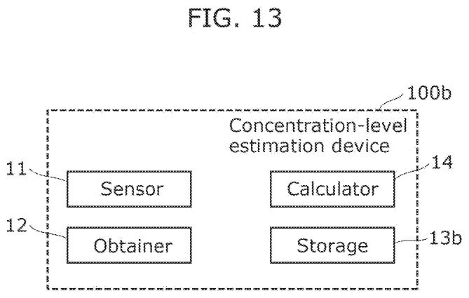

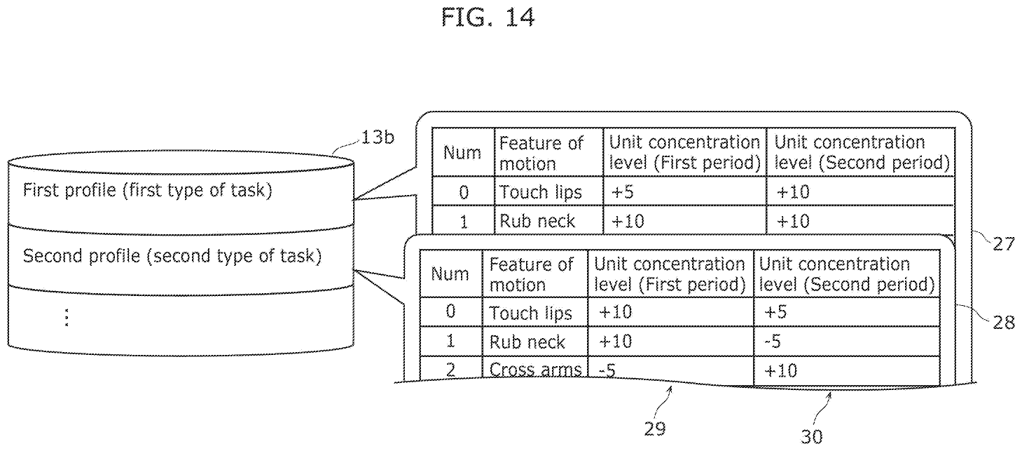

FIG. 13 is a block diagram illustrating the functional configuration of the concentration-level estimation device according to Modification 2 of the embodiment. FIG. 14 is a diagram illustrating the profiles stored in the storage according to Modification 2 of the embodiment.

As illustrated in FIG. 13, the components in concentration-level estimation device 100b according to Modification 2 are not different from those in the concentration-level estimation device 100 according to the embodiment above.

For example, concentration-level estimation device 100b can be used in cases where the habit can be varied due to accumulation of fatigue of user 99 when concentrating, such as when user 99 executes the task for a long time. In concentration-level estimation device 100b according to Modification 2, as illustrated in FIG. 14, storage 13b contains the unit concentration levels corresponding to the features of the motions of user 99 for a first period and a second period different from the first period during which user 99 is executing a task. Storage 13b in the drawing contains first profile 27 used to calculate the concentration level of user 99 when user 99 is executing the first type of task, and second profile 28 used to calculate the concentration level of user 99 when user 99 is executing the second type of task. Here, as described above, the unit concentration levels for the first period and the second period are set in each of first profile 27 and second profile 28.

Hereinafter, the operation will be specifically described using second profile 28. For example, when calculator 14 calculates the concentration level of user 99 in the first period and identifies the action of user 99 as “touch lips” from the received motion information, calculator 14 adds+10 to the concentration level. In contrast, when user 99 is doing the same action in the second period, calculator 14 adds+5 to the concentration level. In other words, compared to the first period, the concentration is reduced in the second period for the feature of the motion of the action “touch lips”.

For example, when calculator 14 calculates the concentration level of user 99 in the first period, and identifies the action of user 99 as “touch hairs” from the received motion information, calculator 14 adds −5 to the concentration level. In contrast, when user 99 is doing the same action in the second period, calculator 14 adds+10 to the concentration level. In other words, compared to the first period, the feature of the motion of the action “touch hairs” in the second period is changed from a habit when user 99 is distracting to a habit when user 99 is concentrating.

Thus, concentration-level estimation device 100b according to Modification 2 can calculate the concentration level of user 99 using profiles each containing first association information 29 in which the feature of each motion of user 99 is associated with the unit concentration level in the first period, and second association information 30 in which the feature of the motion of user 99 is associated with the unit concentration level in the second period.

Each of the profiles according to Modification 2 may contain three or more pieces of association information in which the features of motions are associated with the unit concentration levels, where the period for executing a task is divided into three or more periods including a third period as well as the first period and the second period.

### Effects

As described above, one aspect of concentration-level estimation device 100 according to the present embodiment includes obtainer 12 which obtains task information indicating a type of a task executed by user 99 among types of tasks; sensor 11 which outputs motion information indicating a feature of a motion of user 99 who executes the task, based on a result of sensing obtained from sensing device 101; storage 13 which stores profiles concerning habits of user 99 for the types of tasks; and calculator 14 which calculates a concentration level of the user using the motion information and a profile corresponding to the type of the task indicated in the task information among the profiles stored in storage 13.

Such concentration-level estimation device 100 can calculate the concentration level of user 99 who has different habits for the types of tasks, using the profiles corresponding to the types of tasks. Accordingly, concentration-level estimation device 100 can calculate the concentration level while appropriately identifying the habit which user 99 may make when concentrating by appropriately switching the profile according to the type of the task. Thus, concentration-level estimation device 100 can appropriately calculate the concentration level.

Moreover, for example, concentration-level estimation device 100a may further include sensing device 101.

Thereby, concentration-level estimation device 100a can sense user 99 using sensing device 101 included in concentration-level estimation device 100a. In other words, sensing device 101 other than concentration-level estimation device 100a does not needed to be included, and the concentration level of user 99 can be calculated only with concentration-level estimation device 100a.

Moreover, for example, in concentration-level estimation device 100, in each of the profiles stored in storage 13, features of motions which user 99 may make when concentrating due to the habits may be associated with unit concentration levels indicating concentration levels corresponding to the features of the motions, and calculator 14 may calculate the concentration level of user 99 by adding a unit concentration level associated in a profile corresponding to the type of the task indicated in the task information, according to the feature of the motion of user 99 indicated in the motion information.

Thereby, concentration-level estimation device 100 can calculate the concentration level of user 99 by adding a present unit concentration level. In other words, because calculation in concentration-level estimation device 100 can be simplified, a processing resource for implementing concentration-level estimation device 100 can be minimized and concentration-level estimation device 100 can be simply implemented.

Moreover, for example, at least one of the profiles stored in storage 13b may contain first association information 29 in which the features of the motions which user 99 may make when concentrating due to the habits are respectively associated with the unit concentration levels indicating the concentration levels corresponding to the features of the motions in a first period in which a task is being executed, and second association information 30 in which the features of the motions which user 99 may make when concentrating due to the habits are respectively associated with the unit concentration levels indicating the concentration levels corresponding to the features of the motions in a second period different from the first period in which the task is being executed; and calculator 14 may calculate the concentration level of user 99 using first association information 29 and the motion information in the first period, and may calculate the concentration level of user 99 using second association information 30 and the motion information in the second period.

Thereby, concentration-level estimation device 100b can divide the period in which the task is being executed into the first period and the second period, and can appropriately calculate the concentration level of user 99 in each of the periods. Thus, concentration-level estimation device 100b can more appropriately calculate the concentration level.

Moreover, for example, sensor 11 may obtain, as a result of sensing, an image captured by imaging device 111 included in sensing device 101, may extract the feature of the motion of user 99 who executes the task from the image, and may output the feature of the motion.

Thereby, concentration-level estimation device 100 can calculate the concentration level of user 99 based on the feature of the motion of user 99 extracted from the image.

Moreover, for example, sensor 11 may obtain, as a result of sensing, a voice signal collected by sound collector 112 included in sensing device 101, may extract the feature of the motion of user 99 who executes the task from the voice signal, and may output the feature of the motion.

Thereby, concentration-level estimation device 100 can calculate the concentration level of user 99 based on the feature of the motion of user 99 extracted from the voice signal.

Moreover, for example, the types of tasks may include a first type of task accompanied by an active action of user 99 when executing the task, and a second type of task accompanied by a passive action of the user when executing the task.

Thereby, for each of the two types of tasks accompanied by active actions and tasks accompanied by passive actions, concentration-level estimation device 100 can appropriately calculate the concentration level from the feature of the motion due to the habit which user 99 may make when concentrating.

Moreover, for example, the second type of task may be learning without initiating any action from user 99, by viewing a video when user 99 executes the task, the video being taken in advance.

Thereby, concentration-level estimation device 100 can appropriately calculate the concentration level from the feature of the motion due to the habit which user 99 may make when concentrating, for the learning without initiating any action from user 99, by viewing a video taken in advance.

Moreover, for example, the types of tasks may include a combined type of task in which the task transits to the first type of task at a timing which is preset while the second type of task is being executed, and transits to the second type of the task after a predetermined period has passed; sensor 11 may further obtain a result of sensing about a reaction of user 99 according to the timing, and may output reaction information; and calculator 14 may further calculate the concentration level of user 99 based on the reaction information.

Thereby, for the combined type of task, concentration-level estimation device 100 can appropriately calculate the concentration level from the feature of the motion due to the habit which user 99 may make when concentrating. Moreover, concentration-level estimation device 100 can correct the concentration level using the obtained reaction information when user 99 executes the combined type of task. Thus, concentration-level estimation device 100 can more appropriately calculate the concentration level.

Moreover, for example, a type of task may be a combined type of task in which the task transits to the first type of task at a timing when the concentration level of user 99 becomes lower than a predetermined threshold while user 99 is executing the second type of task.

Thereby, for the combined type of task, concentration-level estimation device 100 can appropriately calculate the concentration level from the feature of the motion due to the habit which user 99 may make when concentrating. Moreover, when the concentration level of user 99 is reduced, concentration-level estimation device 100 can transit the type of task to improve the concentration level. Thus, concentration-level estimation device 100 can more appropriately calculate the concentration level, and can contribute to keep the concentration level of user 99 at a high level.

Moreover, for example, at least one of the profiles stored in storage 13 may contain first subprofile 21 used to calculate a concentration level of a first user contained in a first classification among a plurality of users, and second subprofile 22 used to calculate a concentration level of a second user contained in a second classification among the plurality of users, the second classification being different from the first classification.

Thereby, concentration-level estimation device 100 can calculate the concentration level based on the habit which the user may make when concentrating, using both the type of task and the classification of the user. Thus, concentration-level estimation device 100 can appropriately calculate the concentration level.

Moreover, for example, each of the profiles stored in storage 13 may contain first subprofile 21 used to calculate the concentration level of the first user, and second subprofile 22 used to calculate the concentration level of the second user.

Thereby, concentration-level estimation device 100 can calculate the concentration level based on the habit which the user may make when concentrating, using both of the type of task and the classification of the user. Thus, concentration-level estimation device 100 can appropriately calculate the concentration level.

Moreover, for example, concentration-level estimation device 100a may further include individual identifier 15 which identifies the user as a specific user, and the profiles stored in storage 13a may contain profiles concerning habits of the specific user for each type of task.

Thereby, concentration-level estimation device 100a can estimate the concentration level based on the habit which the specific user may make when concentrating, using the profiles specific to the specific user. Thus, concentration-level estimation device 100a can more appropriately calculate the concentration level.

Moreover, one aspect of the concentration-level estimation method according to the present embodiment may include obtaining step S102 of obtaining task information indicating a type of a task executed by user 99 among types of tasks; sensing step S101 of outputting motion information indicating a feature of a motion of user 99 who executes the task, based on a result of sensing obtained from sensing device 101; and calculating step S104 of calculating the concentration level of user 99 using the motion information and a profile corresponding to the type of task indicated in the task information among profiles concerning habits of the user for the types of tasks.

Thereby, the concentration-level estimation method can provide the same effects as those of concentration-level estimation device 100 above.

Moreover, one aspect of the recording medium according to the present embodiment is a non-transitory computer-readable recording medium on which a program for causing a computer to execute the concentration-level estimation method described above is recorded.

Thereby, the recording medium can provide the same effects as those of concentration-level estimation device 100 above using a computer.

### OTHER EMBODIMENTS

As above, the concentration-level estimation device, the concentration-level estimation method, and the recording medium according to the present disclosure have been described based on the embodiment and the like, but the embodiment should not be construed as limitations to the present disclosure. For example, the present disclosure also covers embodiments obtained by subjecting the embodiment and the like to a variety of modifications conceived by persons skilled in the art, and any combination of components and functions in the embodiment without departing from the gist of the present disclosure.

For example, a learning efficiency estimation device which digitalizes learning efficiency may be implemented using the concentration-level estimation device according to the present disclosure and test results.

Moreover, for example, the concentration level may be replaced by a distraction level, and a distraction-level estimation device which estimates the distraction-level of a user may be implemented.

Moreover, for example, the concentration-level estimation device may further include a task switcher which transits the type of task executed by the user from one type to another type. As described above, when the concentration level of the user calculated by the calculator is lower than a predetermined threshold, first, the task switcher transits the type of the task executed by the user to the first type. The sensor obtains a result of sensing about the reaction corresponding to the transition of the type of the task to the first type by the task switcher, and outputs reaction information. As described above, the term “reaction” here means that the user replies with the voice and action of the user, such as responding and nodding, or performs an operation, e.g., clicks on the GUI displayed as a pop-up window or the like on the screen.

When the reaction information indicating the presence of the reaction from the user is output in such an operation of the concentration-level estimation device, the calculator may perform at least one of correction of the concentration level calculated in the same way as above and update of the profile by correcting the unit concentration level to improve the accuracy of the calculated concentration level.

When the reaction information indicating the absence of the reaction from the user is output in the operation of the concentration-level estimation device, the task switcher may further transit the type of the task executed by the user based on the output reaction information. Specifically, when a reduction in concentration level of the user is correctly calculated and it is estimated that the user is distracting, the concentration level of the user can be improved by transiting the type of the task by the task switcher. For example, the task switcher transits the type of the task to a type of task in which the user is prompted to do exercise to relax the body by reproducing a video. Moreover, for example, the task switcher may transit a type of task to another type of task in which the user views a content of interest by reproducing such a content.

Thus, the task switcher included in the concentration-level estimation device corrects the calculated concentration level and accurately estimates the concentration level of the user, and when the concentration level of the user becomes lower, the task switcher can operate to give an appropriate task to the user to improve the concentration level.

Moreover, for example, the present disclosure can be implemented not only as a concentration-level estimation device, but also a program including, as steps, processings executed by the components in the concentration-level estimation device, and a computer-readable recording medium having the program recorded thereon. The program may be preliminarily recorded on a recording medium, or may be fed to a recording medium through a wide communication network such as the Internet.

In other words, comprehensive or specific aspects described above may be implemented as a system, a device, an integrated circuit, a computer program, or a computer-readable recording medium, or may be implemented with any combination of a system, a device, an integrated circuits, a computer program, and a recording medium.

### INDUSTRIAL APPLICABILITY

The concentration-level estimation device and the like according to the present disclosure are disposed in buildings such as houses, offices, and cram schools and moving bodies such as vehicles, and are used to appropriately calculate the concentration levels of users and the like.

1. A concentration-level estimation device, comprising:
an obtainer which obtains task information indicating a type of a task executed by a user among types of tasks;
a sensor which outputs motion information indicating a feature of a motion of the user who executes the task, based on a result of sensing obtained from a sensing device;
a storage which stores profiles concerning habits of the user for the types of tasks; and
a calculator which calculates a concentration level of the user using the motion information and a profile corresponding to the type of the task indicated in the task information among the profiles stored in the storage.

2. The concentration-level estimation device according to claim 1, further comprising the sensing device.

3. The concentration-level estimation device according to claim 1,
wherein in each of the profiles stored in the storage, features of motions which the user may make when concentrating due to the habits are respectively associated with unit concentration levels indicating concentration levels corresponding to the features of the motions, and
the calculator calculates the concentration level of the user by adding a unit concentration level associated in a profile corresponding to the type of the task indicated in the task information, according to the feature of the motion of the user indicated in the motion information.

4. The concentration-level estimation device according to claim 1,
wherein at least one of the profiles stored in the storage contains:
first association information in which the features of the motions which the user may make when concentrating due to the habits are respectively associated with the unit concentration levels indicating the concentration levels corresponding to the features of the motions in a first period in which a task is being executed; and
second association information in which the features of the motions which the user may make when concentrating due to the habits are respectively associated with the unit concentration levels indicating the concentration levels corresponding to the features of the motions in a second period different from the first period in which the task is being executed, and
the calculator:
calculates the concentration level of the user using the first association information and the motion information in the first period; and
calculates the concentration level of the user using the second association information and the motion information in the second period.

5. The concentration-level estimation device according to claim 1,
wherein the sensor obtains, as the result of sensing, an image captured by an imaging device included in the sensing device, extracts the feature of the motion of the user who executes the task from the image, and outputs the feature of the motion.

6. The concentration-level estimation device according to claim 1,
wherein the sensor obtains, as the result of sensing, a voice signal collected by a sound collector contained in the sensing device, extracts the feature of the motion of the user who executes the task from the voice signal, and outputs the feature of the motion.

7. The concentration-level estimation device according to claim 1,
wherein the types of tasks include:
a first type of task accompanied by an active action of the user when executing the task; and
a second type of task accompanied by a passive action of the user when executing the task.

8. The concentration-level estimation device according to claim 7,
wherein the second type of task is learning without initiating any action from the user, by viewing a video when the user executes the task, the video being taken in advance.

9. The concentration-level estimation device according to claim 7,
wherein the types of tasks include a combined type of task in which the task transits to the first type of task at a timing which is present while the second type of task is being executed, and transits to the second type of the task after a predetermined period has passed,
the sensor obtains a result of sensing about a reaction of the user according to the timing, and outputs reaction information, and
the calculator further calculates the concentration level of the user based on the reaction information.

10. The concentration-level estimation device according to claim 7,
wherein the type of task is a combined type of task in which the task transits to the first type of task at a timing when the concentration level of the user becomes lower than a predetermined threshold while the user is executing the second type of task.

11. The concentration-level estimation device according to claim 7, further comprising:
a task switcher which transits the type of the task executed by the user from one type to another type,
wherein when the concentration level of the user calculated by the calculator is lower than a predetermined threshold:
the task switcher transits the type of the task executed by the user to the first type;
the sensor further obtains the result of sensing about the reaction of the user according to the transition of the type of the task by the task switcher, and outputs reaction information; and
the concentration-level estimation device causes (1) the calculator to further calculate the concentration level of the user based on the reaction information, or (2) the task switcher to further transit the type of the task executed by the user.

12. The concentration-level estimation device according to claim 1,
wherein at least one of the profiles stored in the storage contains:
a first subprofile used to calculate a concentration level of a first user contained in a first classification among a plurality of users; and
a second subprofile used to calculate a concentration level of a second user contained in a second classification among the plurality of users, the second classification being different from the first classification.

13. The concentration-level estimation device according to claim 12,
wherein each of the profiles stored in the storage contains:
the first subprofile used to calculate the concentration level of the first user; and
the second subprofile used to calculate the concentration level of the second user.

14. The concentration-level estimation device according to claim 1, further comprising:
an individual identifier which identifies the user as a specific user,
wherein the profiles stored in the storage contain profiles concerning habits of the specific user for the types of tasks.

15. A concentration-level estimation method, comprising:
obtaining task information indicating a type of a task executed by a user among types of tasks;
outputting motion information indicating a feature of a motion of the user who executes the task, based on a result of sensing obtained from a sensing device; and
calculating a concentration level of the user using the motion information and a profile corresponding to the type of the task indicated in the task information among profiles concerning habits of the user for the types of tasks.

16. A non-transitory computer-readable recording medium on which a program causing a computer to execute the concentration-level estimation method according to claim 15 is recorded.
